(19) United States
(12) Patent Application Publication
(10) Pub. No.: US 2025/0360313 A1
(43) Pub. Date: Nov. 27, 2025

(54) SUPPORT DEVICE FOR A THERPEUTIC DEVICE

(71) Applicants: Fabio FONTANA, Castelfranco Veneto (IT); Gianluca SCARSCIONI, Roma (IT); Giampiero SCARSCIONI, Roma (IT)

(72) Inventors: Fabio FONTANA, Castelfranco Veneto (IT); Gianluca SCARSCIONI, Roma (IT); Giampiero SCARSCIONI, Roma (IT)

(21) Appl. No.: 18/875,142

(22) PCT Filed: Jun. 14, 2023
(86) PCT No.: PCT/IB2023/056158

§ 371 Date: Dec. 14, 2024

(30) Foreign Application Priority Data
Jun. 15, 2022 (IT) 102022000012704

(51) Int. Cl. A61N 1/36; A61N 1/04; A61N 5/06

(52) U.S. Cl. CPC A61N 1/36021; A61N 1/0484; A61N 5/06; A61N 2005/0645; A61N 2005/0659; A61N 2005/0662

(57) ABSTRACT

Support device for a therapeutic device (1) for a patient, which support device comprises a housing part (2) of said therapeutic device (1) and a constraining part (3) for constraining to an operator, the constraining part (3) being ring-shaped and adapted to be worn on the operator's finger, so that the operator can apply the therapeutic effect of the therapeutic device (1) on the patient, wherein the therapeutic device (1) is adapted for the treatment of painful, inflammatory pathologies and for neuro-muscular and postural remodulation of the patient and comprises at least one nano-crystal capable of emitting photons in a wavelength comprised between 400 and 990 nm, when solicited by at least one infrared, luminous or ultraviolet electromagnetic radiation.

The present invention relates to a support device for a therapeutic device for a patient, which support device comprises a housing part of said therapeutic device.

Intervention techniques on the human body are currently known for the prevention, treatment and rehabilitation in the areas of motor skills, superior cortical functions, and visceral functions resulting from pathological events of various etiology, both congenital and acquired.

It is known that such techniques can be extended beyond the area presenting the symptomatology and, considering the body as a whole, can aim to restore and favour, through manipulation, the self-healing and self-regulation processes that are present in the human body.

Recent studies have led to the development of innovative technologies and techniques, which aim to integrate with traditional and modern treatments to increasing the effectiveness thereof, in particular focusing on the treatment of movement disorders and proprioceptive improvement.

Such technology is disclosed in WO 2021/084424 A1, in which therapeutic devices capable of transmitting photons consistent with the human body are described. Such therapeutic devices constantly stimulate the neuralgic points which are imbalanced, without releasing any chemical substance, increase proprioception, and therefore constitute an innovative technology for health and well-being, based on modern knowledge of biophysics applied to health and well-being.

Such therapeutic devices are intended to improve the movement of the body and help the recovery of joint functions, reduce any pain due to incorrect postural patterns, correcting such incorrect postural patterns, thus promoting energy recovery that the body can use for an increase in overall well-being.

Such a therapeutic device is in the form of a small laminar element which is applied to the skin with the help of a bandage tape. This can allow a postural balance to be maintained in the long term, complementing the effects of the treatments under study.

However, for an improved integration of the beneficial effects of these devices with those of therapeutic manipulation by an operator on a patient, it would be appropriate to move the application of these devices over the different areas affected by the manipulation in real time. The aforesaid application by means of a bandage is in this case impractical and of uncertain effectiveness.

There is therefore a need unsatisfied by the state of the art for a support device of a therapeutic device of the aforesaid type, or of similar devices for other functions, which allows the real-time application of the therapeutic device on the patient in the areas affected by the manipulation.

CN110123612A discloses a hand function rehabilitation device, which comprises a plurality of finger rings that can be worn on a finger for a rehabilitation treatment. Each of the rings is provided with a motor for generating vibrations. The hand function rehabilitation device can stimulate the hand and help the patient to carry out the hand rehabilitation treatment. The ring described by this document cannot allow an operator to apply the therapeutic effect on a patient.

U.S. Pat. No. 10,799,425B2 discloses an oral health device, comprising a strip of material to be inserted in the mouth in a manner similar to chewing tobacco and which comprises a dispenser of drugs or other substances, for example with certain tastes. The strip can then be ring-shaped for storing the device on the hand when not in use. Also what is described by this document cannot allow an operator to apply the therapeutic effect on a patient.

WO2021217013A1 discloses an anti-infective light radiation device, configured to provide an output energy of at least one wavelength of antimicrobial electromagnetic radiation within a range of 350 nm-450 nm, therefore it does not solve the technical problems solved by the present invention.

The present invention aims to overcome this limit of the state of the art with a support device as described at the beginning, which further comprises a constraining part for constraining to an operator, which constraining part is ring-shaped and is adapted to be worn on the operator's finger, so that the operator can apply the therapeutic effect of the therapeutic device on the patient. The therapeutic device is adapted for the treatment of painful, inflammatory diseases and for a neuro-muscular and postural remodulation of the patient and comprises at least one nano-crystal capable of emitting photons in a wavelength comprised between 400 and 990 nm, when solicited by at least one infrared, light or ultraviolet electromagnetic radiation.

Thereby the device object of the present invention provides housing for devices generating other types of stimulation to the patient, in addition to the mechanical stimulation offered by the hand of the operator, in particular by means of the emission of photons by at least one nano-crystal.

An increase in the application areas of the therapeutic device is also ensured, and the device object of the present invention represents the possibility for the operator to reach points of the patient's body where the application of some machinery is not possible, for example the scalp, eyes, by means of the eyelids, and others. For this reason, the device is a trans-disciplinary object which can be used in various medical fields.

In an embodiment the housing part comprises a bezel and the constraining part comprises an annular-shaped band, the bezel and the band being connected to each other, and said therapeutic device being coupled to the bezel by retention means.

In a further embodiment, the bezel is provided with a through opening, said therapeutic device being positioned to occlude the opening.

This allows the operator to increase their perception: during the application, the operator's hand is capable of appreciating the change of the patient's tissue, increasing and not hindering the perception itself.

In an embodiment, said opening is of elongated shape along a direction parallel to the insertion direction of the finger in the ring-shaped constraining portion.

Preferably, the entire bezel has an elongated shape corresponding to the opening.

This allows the contact surface on the patient's tissue to be increased while keeping the range of motion of the finger free in biomechanics and during the execution of the manipulation technique. Made in this manner, the form allows inclusion and exclusion of the therapeutic device during the execution of the manipulation technique depending on the flexion-extension of the operator's phalanx.

The through opening in the bezel brings the operator's finger in contact with the therapeutic device, thereby increasing the operator's sensitivity and perception.

According to a preferred embodiment, said opening is oval in shape.

This shape is particularly beneficial for the use of the therapeutic device on the operator's fingertips, maintaining all tactile sensitivity during the manipulation. The operator can therefore move the support device along the finger during the manipulation according to the therapeutic needs, always having a wide contact surface of the therapeutic device with the skin and an optimized sensitivity of the patient's tissue feedback.

According to an embodiment, said band is open.

This improves comfort, fixing and adaptability on the finger and increases the ability of the device to be worn by multiple sizes, even on the same finger or on the same hand, without altering the sensitivity of the operator.

According to a further embodiment, said band is divided into two parts having two respective end zones, or tails, arranged side by side.

This allows to increase the contact surface of the band with the operator's finger, increasing the antero-posterior fixing during the manipulations.

According to an embodiment, the therapeutic device comprises at least one supporting laminar element of said at least one nano-crystal, a first side of the supporting element being adapted to be associated in contact with the skin of the patient and at least one said nano-crystal being on a second side, opposite said first side of the supporting element, placed, or incorporated in the supporting element itself.

The use of these therapeutic devices in combination with the support device according to the present invention has been surprisingly effective in therapeutic manipulations, in particular those aimed at neuro-muscular and postural remodulation.

These and other features and advantages of the present invention will become clearer from the following description of some embodiments illustrated, by way of example only, in the attached drawings, in which:

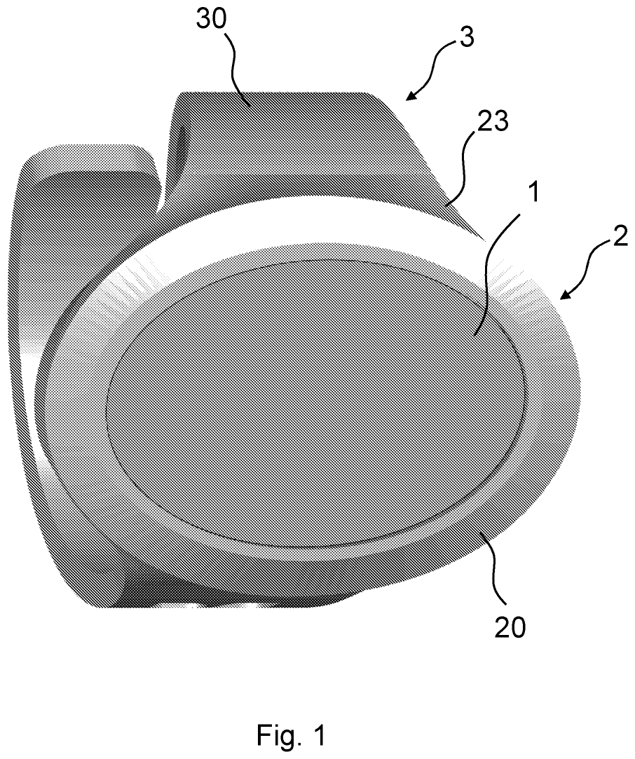

FIG. 1 illustrates an embodiment of the support device, with the therapeutic device coupled to the housing part;

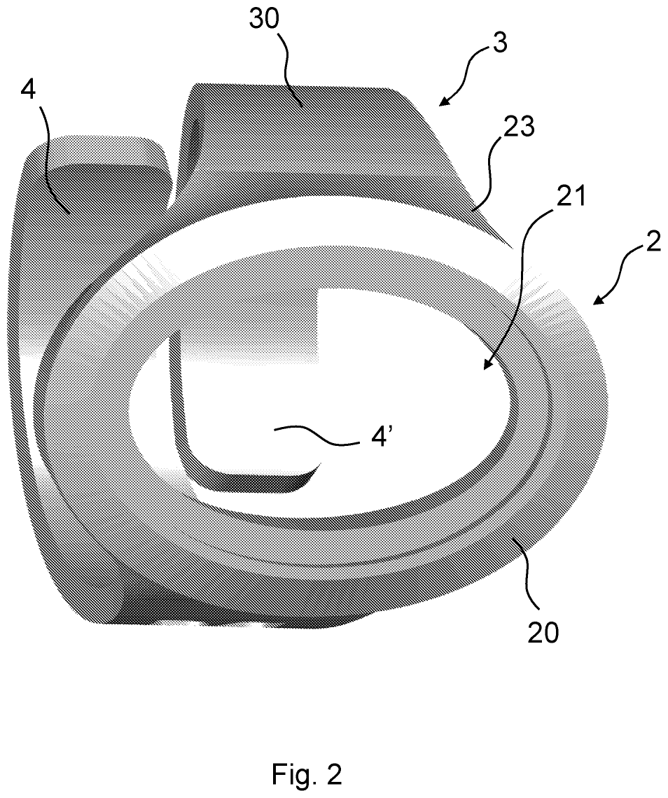

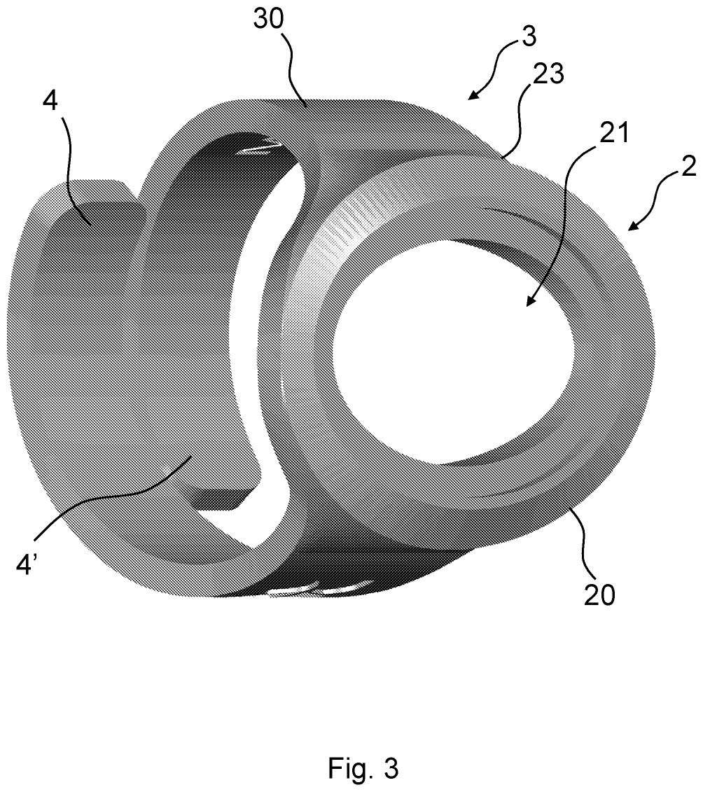

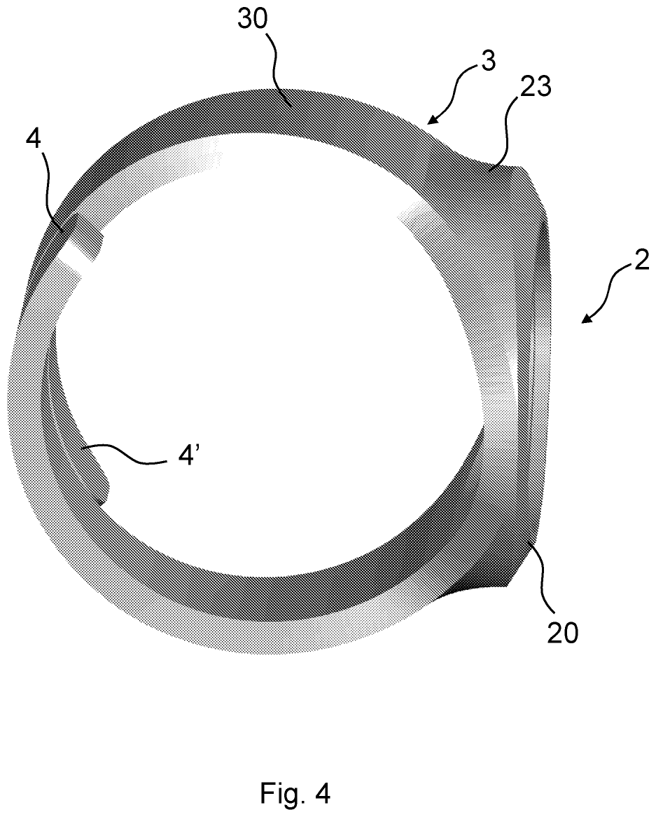

FIGS. 2 to 4 illustrate different views of the embodiment of the support device, without the therapeutic device.

The figures show an embodiment of the support device object of the present invention, configured to carry a therapeutic device 1.

The therapeutic device 1 may be of any type, preferably it is adapted to be applied on a patient by an operator.

The support device comprises a housing part 2 of the therapeutic device 1 and a constraining part 3 for constraining to an operator. The constraining part 3 is ring-shaped and is adapted to be worn on the operator's finger.

The housing part 2 comprises a bezel 20 and the constraining part comprises an annular-shaped band 30, the bezel 20 and the band 30 being connected to each other, and said therapeutic device 1 being coupled to the bezel 20 by retention means.

The retention means may be of any type known in the state of the art, for example gluing or partial incorporation in the material of the bezel.

The bezel 20 is provided with a through opening 21 and the therapeutic device 1 is placed at least partially occluding the opening 21.

Preferably, the opening 21 is of elongated shape along a direction parallel to the insertion direction of the finger in the ring-shaped constraining part 3, and advantageously is of oval shape.

In the embodiment illustrated in the figures, the therapeutic device 1 is advantageously of corresponding shape, preferably oval, and completely occludes the opening 21. To this end, the bezel 20 is provided, at the edges of the opening 21, with an oval-crown shoulder for supporting and fixing the peripheral zone of the therapeutic device 1.

However, it is possible to provide a bezel 20 without a through opening 21, for example full. In this case the bezel 20 has a face facing the opposite direction than the rest of the ring, the therapeutic device 1 being fixed on such a face. Also in this case it is possible to provide an elongated shape of the bezel 20 and of the therapeutic device 1, preferably oval.

The junction between the band 30 and the bezel 20 forms the abutment 23.

The band 30 is internally smooth and has an enlargement, along the insertion direction of the operator's finger, near the abutment 23. This increases the structural solidity of the entire device, increases the compactness thereof and improves the fit thereof on the operator's finger. In the internal face of the bezel 20, the band 30 leaves room for the opening 21, ending with an internal groove which further increases the contact space between the operator's finger and the therapeutic device 1.

The external part of the band 30 follows the internal band in the structural trend and, converging laterally to the bezel 20, creates the abutment 23.

Advantageously, the band 30 is open, being divided into two parts fixed to the bezel 20 by means of the abutments 23. Such two parts of the band 30 have two respective end zones 4, 4′ facing each other along the insertion direction of the operator's finger. For a flanking section of the end zones 4, 4′ of predetermined angular extension, therefore, the band 30 preferably has a substantially double width with respect to the remaining zones.

Alternatively, however, it is possible to provide a closed annular band.

The band 30 and the bezel 20 are preferably in a single piece, but may consist of individual elements fixed together with fixing means known to those skilled in the art.

The material of the band 30 and the bezel 20 may be of any type, preferably polymeric.

The therapeutic device is adapted for the treatment of painful, inflammatory diseases and for a neuro-muscular and postural remodulation of a patient and comprises at least one nano-crystal capable of emitting photons in a wavelength comprised between 400 and 990 nm, when solicited by at least one infrared, light or ultraviolet electromagnetic radiation.

Such a therapeutic device 1 comprises at least one supporting laminar element of said at least one nano-crystal, a first side of the supporting element being adapted to be associated in contact with the skin of the patient and at least one said nano-crystal being on a second side, opposite the first side of the supporting element, placed, or incorporated in the supporting element itself.

The laminar element is made of transparent material at the aforementioned reference wavelengths and at least one infrared, luminous, ultraviolet electromagnetic radiation.

Preferably a mixture of nano-crystals is placed on the laminar element or incorporated or diffused in the laminar element itself, with a concentration from 1 mg/cm² to 6 g/cm² and capable of emitting photons in the reference wavelength.

The nano-crystal or mixture of nano-crystals comprises one or more of the following quantum dots:
Graphene quantum dots code 900708 or quantum dots with a fluorescence indicatively corresponding to: λex 350 nm; λem 445 nm, FWHM 65 nm, quantum yield >65%;
Blue luminescent graphene quantum dots, code 900726 or quantum dots with a fluorescence indicatively corresponding to: λex 350 nm; λem 445 nm+10 nm, FWHM 75 nm, quantum yield >20%
Cyan luminescent graphene quantum dots, code 900707 or quantum dots with a fluorescence indicatively corresponding to: λem 475-495 nm, FWHM 70 nm, quantum yield >17%
Aqua green luminescent graphene quantum dots, code 900712 or quantum dots with a fluorescence indicatively corresponding to: λex 485 nm; λem 530 nm+10 nm, FWHM 80 nm, quantum yield >17%;
Perovskite quantum dots coated with oleic acid and oleylamine, code 900747 or quantum dots with a fluorescence approximately corresponding to: λem 480 nm;
COOH functionalised core-type CdTe quantum dots, code 777978 or quantum dots with a fluorescence indicatively corresponding to: λem 710 nm, quantum yield >15%;
CdS/ZnS functionalised oleic acid core-shell type quantum dots, code 900286 or quantum dots with a fluorescence indicatively corresponding to: λmax 385 nm λem 400 nm+10 nm, quantum yield >50%;
CdS/ZnS functionalised oleic acid core-shell type quantum dots, code 900283 or quantum dots with a fluorescence approximately corresponding to: Amax 405 nm λem 425 nm+10 nm;

The nano-crystals or the mixture of nano-crystals allows to reach and produce the same wavelengths as ULLLT (ultra-low-level laser therapy) but with ultra-weak intensity.

The laminar element consists of a plastic material capable of associating with the patient's epidermis, being of flexible material capable of associating with the patient's epidermis and of following the movements and/or deformations thereof without detaching and being waterproof and inert with respect to the sweat emitted from the skin.

The laminar element has a thickness comprised between 0.05 mm and 2 mm, and even more preferably comprised between 0.1 and 1 mm, being better able to adapt to the deformations of the epidermis.

A second laminar element for the protection and confinement of the layer of nano-crystals is provided, consisting of a transparent material either with infrared radiation or with light radiation in visible or ultraviolet light, so that the radiation coming from outside and passing through the second laminar element can excite the aforesaid mixture of nano-crystals to emit the frequencies of interest.

1. A support device for a therapeutic device for a patient, which support device comprises a housing part of said therapeutic device,
wherein the support device comprises a constraining part for constraining to an operator, the constraining part being ring-shaped and adapted to be worn on the operator's finger, so that the operator can apply the therapeutic effect of the therapeutic device on the patient, wherein the therapeutic device is adapted for the treatment of painful, inflammatory pathologies and for neuro-muscular and postural remodulation of the patient and comprises at least one nano-crystal capable of emitting photons in a wavelength comprised between 400 and 990 nm, when solicited by at least one infrared, luminous or ultraviolet electromagnetic radiation.

2. The support device according to claim 1, wherein the housing part comprises a bezel and the constraining part comprises a band of annular shape, the bezel and the band being connected to each other, and said therapeutic device being coupled to the bezel by retention means.

3. The support device according to claim 1, wherein the bezel is provided with a through opening, said therapeutic device being placed to occlude the opening.

4. The support device according to claim 3, wherein said opening is elongated along a direction parallel to the insertion direction of the finger in the ring-shaped constraining portion.

5. The support device according to claim 4, wherein said opening is oval in shape.

6. The support device according to claim 2, wherein said band is open.

7. The support device according to claim 6, wherein said band is divided into two parts having two respective end zones arranged side by side.

8. The device according to claim 1, wherein the therapeutic device comprises at least one supporting laminar element of said at least one nano-crystal, a first side of the supporting element being adapted to be associated in contact with the skin of the patient and at least one said nano-crystal being on a second side, opposite said first side of the supporting element, placed, or incorporated in the supporting element itself.
